(19) United States
(12) Patent Application Publication
(10) Pub. No.: US 2026/0137746 A1
(43) Pub. Date: May 21, 2026

(54) Room-Temperature Cannabis/Hemp Extraction System with Custom Membrane Filtration for Enhanced Yield, Purity, and Energy Efficiency

(71) Applicant: Kameron Walker, Rock Springs, WY (US)

(72) Inventor: Kameron Walker, Rock Springs, WY (US)

(21) Appl. No.: 18/953,190

(22) Filed: Nov. 20, 2024

(51) Int. Cl. A61K 36/185; A61K 31/00; B01D 11/02

(52) U.S. Cl. CPC A61K 36/3482; A61K 31/658; B01D 11/0288; A61K 2236/15; A61K 2236/333; A61K 2236/39; A61K 2236/51

(57) ABSTRACT

The present invention provides a novel room-temperature extraction system and method for cannabis and hemp, utilizing custom membrane filtration to efficiently isolate cannabinoids, terpenes, and other bioactive compounds while reducing energy consumption. This multi-stage system begins with a custom milling machine that preprocesses biomass by separating non-target components, such as cellulose-rich stalks, to improve extraction efficiency. The core extraction process employs a series of custom membranes designed to selectively filter cannabinoids and terpenes, minimizing impurities without the need for traditional sub-zero cooling. Each membrane stage incorporates solvent recovery to recycle ethanol, reducing waste and operational costs. In the final stage, a custom concentrator removes residual ethanol, decarboxylates THCa to THC, and crystallizes sugars to prevent downstream clogging. This room-temperature extraction system significantly enhances yield, purity, and sustainability, making it a scalable and cost-effective solution for cannabis and hemp processing industries, as well as applicable for pharmaceutical and nutraceutical uses.

### BACKGROUND OF THE INVENTION

### Field of Invention

This invention pertains to the field of botanical extraction systems with a particular focus on cannabis and hemp industries, aiming to improve the efficiency, purity, and sustainability of cannabinoid and terpene extraction. Traditional extraction systems depend on sub-zero cooling to prevent impurities from co-extracting with target compounds, resulting in high energy costs and operational complexities. The invention addresses these challenges by integrating a room-temperature extraction process, utilizing custom membrane filtration to selectively separate and concentrate cannabinoids, terpenes, and other valuable bioactive compounds, without the need for extensive cooling. This approach not only enhances extract purity and yield but also reduces operational costs by minimizing energy consumption.

Beyond standard extraction, the system introduces a unique series of stages, including custom milling, filtration, decarboxylation, and solvent recovery, each specifically optimized for the structural and chemical properties of cannabis and hemp biomass. By focusing on energy-efficient extraction that meets high purity standards, this invention is positioned as a transformative solution for industries reliant on botanical extracts, including pharmaceuticals, nutraceuticals, and wellness products. This technology offers a scalable, cost-effective, and environmentally sustainable alternative to conventional extraction methods, potentially setting a new standard in the field.

### BRIEF SUMMARY OF THE INVENTION

The invention provides a room-temperature extraction system for cannabis and hemp biomass, leveraging custom membrane filtration technology to enhance extraction efficiency, purity, and yield without the need for traditional cooling. By operating at room temperature, the system reduces energy consumption and lowers operational costs. This multi-stage system includes a custom milling machine to prepare biomass, removing unnecessary plant components, and a series of membrane filtration stages that selectively separate cannabinoids and terpenes while recovering solvents.

A unique concentrator in the final stage removes residual ethanol, decarboxylates THCa to THC, and crystallizes sugars to prevent clogging in downstream processes. Designed for scalability and sustainability, this invention is particularly suited for applications in the cannabis/hemp, pharmaceutical, and nutraceutical sectors, providing a cost-effective and environmentally friendly alternative to existing extraction technologies.

### BRIEF DESCRIPTION OF THE FIGURES

FIG. 1: illustrates a multi-stage process utilizing the extraction system that combines custom milling, sequential room-temperature membrane filtration, solvent recovery, and concentration to produce a high-purity cannabis/hemp extract with minimized impurities, reduced energy consumption, and enhanced yield.

FIG. 2 illustrates a first stage of a system according to an embodiment of the present invention

FIG. 3 illustrates another stage of a system according to an embodiment of the present invention

FIG. 4 illustrates another stage of a system according to an embodiment of the present invention

FIG. 5 illustrates another stage of a system according to an embodiment of the present invention

FIG. 6 illustrates another stage of a system according to an embodiment of the present invention

### DETAILED DESCRIPTION

The present invention provides a proprietary, multi-stage extraction system for the efficient, high-purity extraction of cannabinoids, terpenes, and other bioactive compounds from cannabis and hemp biomass. Unlike traditional methods that rely on energy-intensive cooling to sub-zero temperatures to prevent impurities from co-extracting with the desired compounds, this system operates entirely at room temperature. This reduction in energy consumption lowers operational costs and reduces environmental impact. Designed for the cannabis, hemp, pharmaceutical, and nutraceutical sectors, the invention improves extract quality, enhances scalability, and addresses common issues such as solvent waste and product purity.

The invention initiates the extraction process with a custom milling machine designed to preprocess cannabis/hemp biomass. This machine grinds the entire plant while removing stalks, which are high in cellulose and low in target compounds, such as cannabinoids and terpenes. Traditional extraction methods often do not adequately preprocess biomass, resulting in excess cellulose and plant debris entering the extraction phase. This unwanted plant material can reduce extraction efficiency and cause clogging and fouling in filters and membranes, complicating further processing. By specifically removing stalks and preparing the biomass at this stage, the system minimizes interference from cellulose and unwanted plant matter, improving the extraction process's selectivity and reducing downstream maintenance and processing interruptions. This preprocessing step directly impacts the final product quality by increasing the concentration of cannabinoids and terpenes relative to non-beneficial plant compounds, yielding a cleaner extract from the outset.

Following the milling process, the invention employs custom membrane filtration stages, which serve as the core of the room-temperature extraction system. These filtration stages utilize proprietary membrane materials and configurations that selectively isolate and concentrate cannabinoids and terpenes while blocking impurities, including chlorophyll, waxes, and other undesirable compounds. Unlike conventional cooling-based extraction methods that achieve selectivity by lowering temperatures to inhibit the solubility of impurities, this membrane-based approach allows selectivity at ambient temperatures through chemical engineering of the membrane's pore sizes, surface charge, and structural materials. Each membrane is optimized to capture compounds based on molecular size and affinity, thereby increasing the efficiency and purity of the extraction process. By filtering out impurities in a controlled, stepwise manner, the system achieves a high concentration of cannabinoids and terpenes with fewer impurities, resulting in a cleaner, more concentrated final product. Additionally, this membrane filtration process recovers solvents at each stage, significantly reducing waste and lowering overall solvent costs, which directly impacts operational expenses.

The final stage of the invention includes a custom concentrator unit specifically designed to finalize the extraction process by removing residual ethanol, decarboxylating THCa into Delta-9 THC, and crystallizing sugars to prevent clogging in downstream equipment. The removal of ethanol at this stage is critical, as residual solvents can dilute the product and negatively impact purity. Traditional methods require additional equipment for decarboxylation, which increases the processing time and operational complexity. By integrating decarboxylation directly within the concentrator unit, the invention not only streamlines the workflow but also provides controlled thermal exposure that maximizes THC yield without degrading other compounds. Moreover, the crystallization of sugars within this unit prevents clogging in the wiped-film distillation process, which is typically used to finalize cannabinoid products. In conventional systems, uncrystallized sugars lead to frequent clogs, requiring system downtime and maintenance. The crystallization stage thus enhances process stability and uptime, increasing throughput and minimizing maintenance costs.

The invention further includes a solvent recovery system designed to work synergistically with the room-temperature extraction setup. Traditional extraction processes often waste solvents, leading to high material costs and environmental waste. In this system, solvent recovery is tightly integrated with the extraction process to allow efficient recapture of ethanol, significantly reducing the need for fresh solvent input and lowering both costs and environmental impact. The solvent recovery system operates without additional cooling requirements, unlike conventional systems, which often rely on extensive cooling infrastructure. This system-wide solvent recapture capability ensures an efficient, sustainable, and cost-effective operation by reducing the environmental burden associated with large-scale solvent use and minimizing ongoing operational expenses.

### Impact of the Invention Over Traditional Methods

This novel system provides a robust alternative to traditional extraction methods by enhancing the purity, yield, and scalability of cannabinoid and terpene extraction. By operating at room temperature, it circumvents the limitations of temperature-sensitive extractions, offering a cleaner, more sustainable option. The selective membrane filtration process uniquely addresses impurities without the energy costs associated with cooling, yielding a purer extract with minimal undesired compounds. This improvement in purity translates to a higher-quality product, particularly beneficial for medical and pharmaceutical applications where compound purity is critical.

The present invention addresses key challenges in cannabinoid extraction, providing a sustainable and scalable method that enhances product purity and consistency, reduces operational costs, and minimizes environmental impact. By improving on traditional extraction methods, this invention stands to set a new industry standard for energy-efficient and high-quality cannabis and hemp extraction processes.

### Detailed Description of Figures

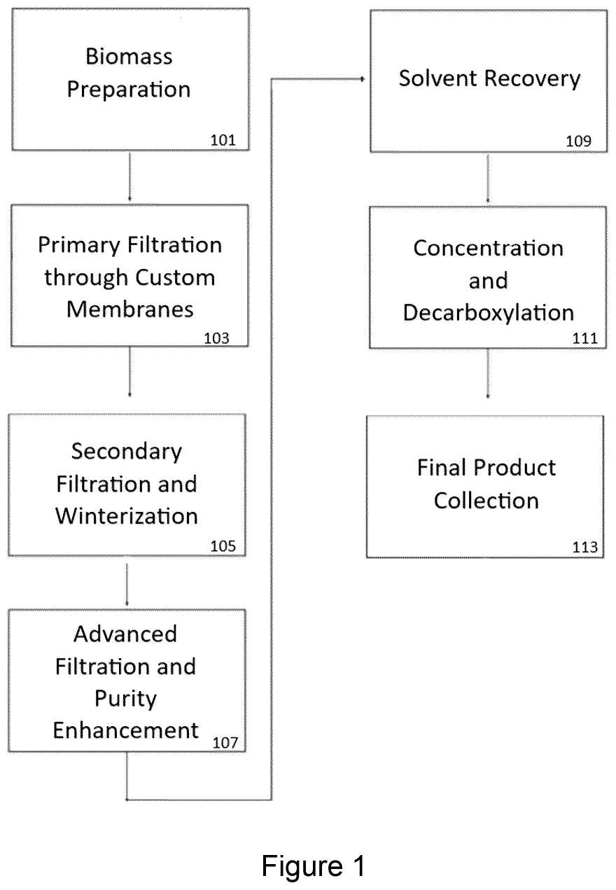

FIG. 1.101: Biomass Preparation—The process begins with biomass preparation, where cannabis or hemp is fed into a custom milling machine. This machine grinds the entire plant, including stalks, leaves, and buds, then separates stalks from the rest of the material. Stalks are rich in cellulose and contain low cannabinoid concentrations, which can interfere with extraction efficiency and purity. Removing these cellulose-rich components enhances the selectivity of the extraction and reduces clogging in downstream filtration stages. This preprocessing step results in a higher concentration of cannabinoids and terpenes in the target biomass.

FIG. 1.103: Primary Filtration through Custom Membranes—The milled biomass undergoes the first filtration stage using custom-designed membranes that operate at room temperature. These membranes selectively isolate cannabinoids and terpenes by size and chemical affinity, blocking chlorophyll and other unwanted plant compounds. Unlike traditional cooling methods that reduce impurity solubility, this room-temperature filtration enables a selective and energy-efficient extraction. By the end of this stage, the initial extract has been enriched in cannabinoids and terpenes while minimizing impurities, streamlining the subsequent purification steps.

FIG. 1.105: Secondary Filtration and Winterization—In the second filtration stage, a series of membranes provide additional separation, targeting residual impurities such as plant waxes. These waxes, typically removed by low-temperature winterization, are effectively separated without cooling in this system, thanks to the specialized membrane properties. This step improves the extract's purity while maintaining the cannabinoids and terpenes concentration. The absence of traditional cooling requirements reduces energy consumption, making this winterization alternative more sustainable and cost-effective.

FIG. 1.107: Advanced Filtration and Purity Enhancement—The extract then enters a tertiary filtration stage, which focuses on purity enhancement. Here, the membrane system achieves higher selectivity, further concentrating cannabinoids and removing trace impurities. This advanced filtration stage produces an extract of superior quality, with high cannabinoid and terpene purity suitable for pharmaceutical and nutraceutical applications. This high-purity extract minimizes the need for additional refinement, reducing processing time and cost.

FIG. 1.109: Solvent Recovery—After each filtration stage, the system integrates a solvent recovery process, capturing and recycling ethanol. Ethanol recovery reduces solvent waste and overall operational costs. Recovered ethanol is cycled back into the process, maintaining extraction efficiency and lowering the environmental impact. Unlike traditional methods requiring complex cooling, this room-temperature system's solvent recovery operates effectively without additional cooling infrastructure, making it both economical and environmentally friendly.

FIG. 1.111: Concentration and Decarboxylation—The concentrated extract is processed in a custom concentrator, where residual ethanol is removed, cannabinoids undergo decarboxylation (converting THCa to active THC), and sugars crystallize to prevent clogging in downstream processing. The controlled decarboxylation process ensures maximum THC yield without degrading other compounds. Crystallization of sugars at this stage improves the system's stability by preventing blockages in subsequent distillation steps, reducing maintenance needs and enhancing throughput.

FIG. 1.113: Final Product Collection—The final, purified extract is collected, containing a high concentration of cannabinoids and terpenes with minimal impurities. This extract is now ready for further processing or formulation, suitable for a range of applications in cannabis and hemp product manufacturing, including pharmaceutical and nutraceutical formulations. The room-temperature system's high yield, purity, and operational efficiency make this extract particularly valuable for high-quality end products.

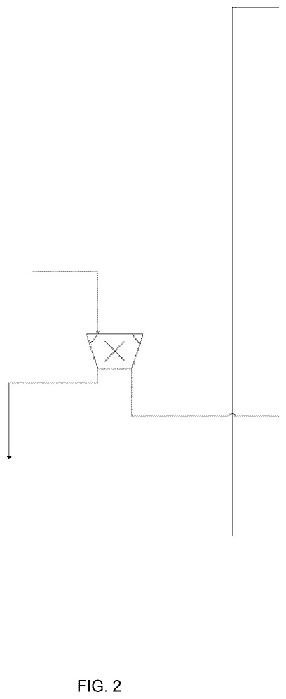

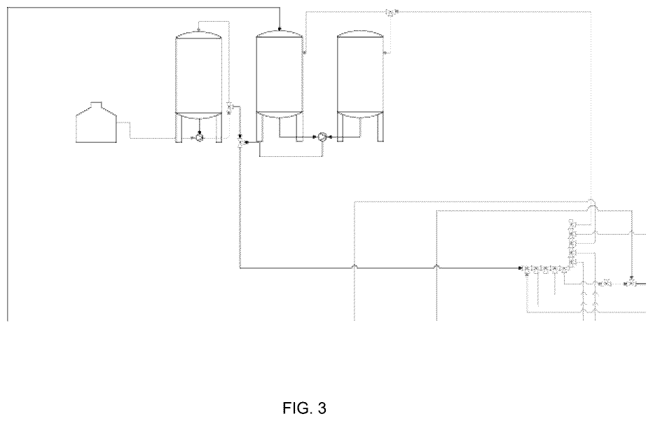

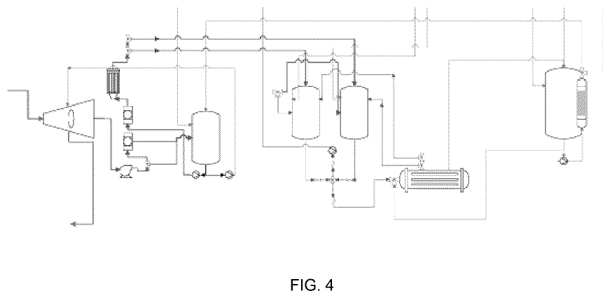

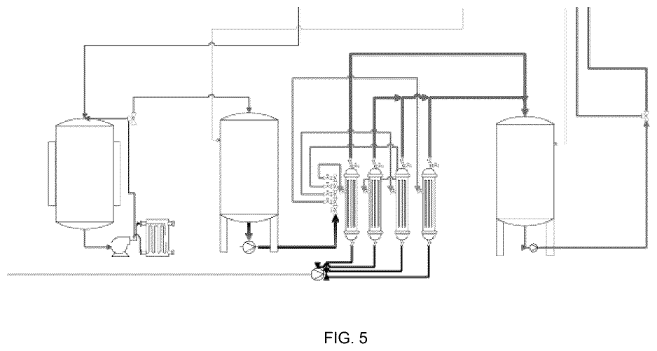

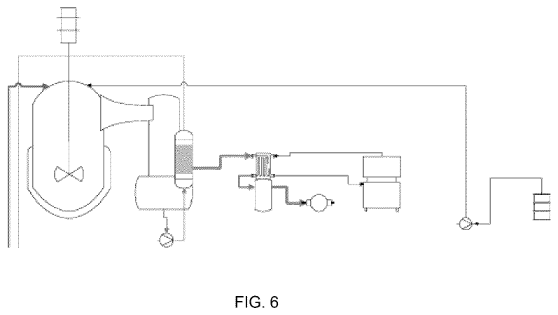

FIGS. 2-6 illustrate stages of the system for performing the process just described.

What is claimed is:

1. A method for extracting cannabinoids, terpenes, and other bioactive compounds from cannabis or hemp biomass at room temperature, comprising the steps of:
a. (a) Grinding and separating biomass into target and non-target components with a custom milling machine, wherein cellulose and non-essential plant material are removed to enhance extraction efficiency;
b. (b) Subjecting the milled biomass to a series of membrane filtration stages operating at room temperature, where each membrane is configured to selectively separate and concentrate cannabinoids and terpenes while blocking unwanted impurities, including chlorophyll and waxes;
c. (c) Implementing solvent recovery at each filtration stage to capture and recycle ethanol, reducing solvent waste and operational costs;
d. (d) Concentrating the miscella in a custom concentrator, wherein residual ethanol is removed, cannabinoids are decarboxylated to transform THCa to THC, and sugars are crystallized to prevent clogging in subsequent wiped-film distillation; and
e. (e) Collecting the purified cannabis or hemp extract.

2. The method of claim 1, wherein the membrane filtration stages are designed with specific pore sizes and chemical properties that allow cannabinoids and terpenes to pass through while preventing chlorophyll, waxes, and other impurities from entering the final extract.

3. The method of claim 1, wherein solvent recovery at each filtration stage is achieved by a recycling process that minimizes solvent loss without requiring cooling infrastructure.

4. The method of claim 1, wherein the decarboxylation in step (d) occurs within the concentrator through controlled heating, enabling the conversion of THCa to Delta-9 THC without additional heating equipment.

5. A custom milling machine for preprocessing cannabis or hemp biomass for extraction, comprising:
a. (a) A grinding mechanism configured to mill the entire biomass; and
b. (b) A separation mechanism that removes non-target plant material, including stalks high in cellulose, while preserving cannabinoids and terpenes in the target biomass, thereby enhancing extraction selectivity and efficiency.

6. The custom milling machine of claim 5, wherein the separation mechanism is designed to reduce clogging and fouling in downstream membrane filtration processes by removing cellulose and other unwanted plant materials.

7. (canceled)

8. (canceled)

9. A custom concentrator for processing cannabis or hemp extracts, comprising:
a. (a) An ethanol removal unit to eliminate residual ethanol from the extract;
b. (b) A decarboxylation unit integrated within the concentrator to convert THCa to Delta-9 THC at controlled temperatures;
c. (c) A sugar crystallization system that crystallizes sugars within the extract, preventing clogging in downstream wiped-film distillation processes; and
d. (d) A heating and control system that maintains a temperature profile conducive to decarboxylation without degrading other active compounds.

10. The custom concentrator of claim 9, wherein the decarboxylation unit allows for precise temperature control to avoid excessive heat exposure, maintaining cannabinoid integrity during the conversion process.

11. The custom concentrator of claim 9, wherein the crystallization system is configured to reduce blockages and maintenance requirements in downstream processes, increasing throughput and process efficiency.

12. The method of claim 1, further comprising an integrated control system that automates and monitors each extraction stage, optimizing extraction parameters for temperature, solvent recovery, filtration, and decarboxylation to achieve maximum yield and purity.
